(19) United States
(12) Patent Application Publication
(10) Pub. No.: US 2005/0069519 A1
(43) Pub. Date: Mar. 31, 2005

(54) Marine growth preventive agent

(75) Inventors: Yoshihito Osada, Sapporo-shi (JP); Jian Ping Gong, Sapporo-shi (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: HOKKAIDO TECHNOLOGY LICENSING OFFICE CO., LTD, Sapporo-shi (JP)

(21) Appl. No.: 10/504,404

(22) PCT Filed: Feb. 18, 2002
(86) PCT No.: PCT/JP02/01356

(51) Int. Cl. A61K 31/74

(52) U.S. Cl. 424/78.09

(57) ABSTRACT

An anti-adhesion agent for marine organisms comprising a hydrogel, which exerts no influences on the ecosystem or natural environment.

### TECHNICAL FIELD

The present invention relates to an anti-adhesion agent for marine organisms for preventing marine organisms from attaching to fishnets or the bottom of a ship.

### BACKGROUND ART

Currently, a chemical, called anti-fouling agent, is applied to fishnets or the bottom of a ship in order to prevent attachment of fouling organisms. Such a chemical is mainly composed of an herbicidal organic nitrogen- or sulfur- or inorganic copper-based compound so that the compound may gradually elute upon immersion in seawater to show herbicidal efficacy. It means that the chemical may have influences not only on the surfaces of the fishnets, etc. but indiscriminately on beneficial seaweeds such as kelps and edible marine products such as sea urchins and abalones inhabiting in the vicinity as well. In fact, as a result of continued use of such a chemical, a large number of mutations that female shellfish develop the male genital organs can now be evidenced along the coastal areas throughout Japan. In recent years, such anti-fouling agents have constantly been improved in the light of low-impactness on the environment and persistence of the efficacy. In order to be effective to those of shellfish that are highly resistant to toxicants, however, a certain degree of toxicity will unavoidably be needed. Because of their huge consequent influences on the ecosystem, however, use of anti-fouling agents of slow-release type that can be responsible for the marine contamination should permanently be prohibited.

Thus, the present invention aims to provide an anti-fouling process to take the place of prior art anti-fouling agents with no influences on the ecosystem and natural environment.

### DISCLOSURE OF THE INVENTION

The present invention (1) is an anti-adhesion agent for marine organisms which comprises a hydrogel.

Also, the present invention (2) is the anti-adhesion agent according to the invention (1), wherein the hydrogel has a proton concentration of 10⁻⁴ mol/L to 5 mol/L.

Further, the present invention (3) is the anti-adhesion agent according to the invention (2), wherein the proton is derived from an acidic group of a network macromolecule comprising the hydrogel and/or from an acidic substance existing in the gaps in the network macromolecule.

Also, the present invention (4) is the anti-adhesion agent according to the invention (3), wherein the acidic group is selected from the group consisting of a carboxyl group, a hydroxyl group attached to an electron-withdrawing aromatic ring, a sulfonic group and a phosphate group.

In addition, the present invention (5) is the anti-adhesion agent according to the invention (4), wherein the hydrogel is selected from the group consisting of homopolymerized or copolymerized macromolecular gels, chemically or physically containing in their networks, units of poly-2-acrylamido-2-methylpropanesulfonic acid (PAMPS), polyvinylphenol, polymaleic acid, polyacrylic acid (PAA) or polymethacrylic acid (PMAA).

Also, the present invention (6) is the anti-adhesion agent according to any one of the inventions (2) to (5), wherein the acidic substance is an organic or inorganic acid.

### BRIEF DESCRIPTION OF THE DRAWINGS

FIG. 1 shows the status of laminaria angusta zoospores (after two weeks) on PAMP gels having different degrees of swelling (q), wherein (1) q=16, (2) q=12, (3) q=5 and (4) q=3;

FIG. 2 shows the status of laminaria angusta zoospores (after two weeks) on PAA gels having different degrees of swelling (q), wherein (1) q=16, (2) q=12, (3) q=5 and (4) q=3.

FIG. 3 shows the status of laminaria angusta zoospores (after two weeks) on a PMAA gel;

FIG. 4 shows the relationship between the degrees of swelling of PAMPS and PAA gels and the germination rate of laminaria angusta zoospores, wherein ∘ and ● denote the results on the PAMPS and PAA gels respectively; and

FIG. 5 shows the viability of laminaria angusta zoospores using Neutral Red, wherein (1) and (2) denote a heat-treated sample and an untreated sample respectively after four weeks on PAA gels.

### BEST MODE FOR CARRYING OUT THE INVENTION

The hydrogel according the present invention may be any hydrogel that has water as a dispersing medium and may be a synthetic macromolecular gel or a naturally occurring macromolecular gel. A hydrogel whose degree of swelling is 1.5 to 500 is suitable and a hydrogel whose degree of swelling is 2 to 200 is particularly suitable. The term “degree of swelling” as used herein means the total of the weight of water in a hydrogel and the weight of a polymer divided by the weight of the polymer.

As a particularly suitable hydrogel, a hydrogel can be mentioned whose proton concentration is 10⁻⁴ mol/L to 5 mol/L, most preferably 10⁻² mol/L to 1 mol/L. The proton here may be derived from an acidic group of network macromolecule comprising the hydrogel or from an acidic substance existing in the gaps in the network macromolecule, or both. The term “proton concentration” as used herein is the number of mols of the proton that the acidic group or the acidic substance originally possessed divided by the volume of the hydrogel, that is, the total of the number of mols of the proton dissociated from the acidic group or the acidic substance and the number of mols of the proton not dissociated from and still attached to the acidic group or the acidic substance divided by the volume of the hydrogel.

Examples of acidic groups of network macromolecules comprising hydrogels include a carboxyl group, a sulfonic group, a phosphate group, a hydroxyl group attached to an electron-withdrawing aromatic ring (a phenolic group, for example), particularly suitable among them being a carboxyl group. Examples of hydrogels in which network macromolecules have such acidic groups include homopolymerized or copolymerized macromolecular gels, chemically or physically containing in their networks, units of poly-2-acrylamido-2-methylpropanesulfonic acid (PAMPS), polyacrylic acid (PAA), polymethacrylic acid (PMAA), polyvinylphenol or polymaleic acid. The term “chemically” as used herein means being covalently incorporated directly into the network while the term “physically” as used herein means being not directly linked covalently to the network but existing in the gaps of the network.

The acidic substance may be an organic or inorganic acid and of a low molecular weight or high molecular weight. Examples of inorganic acids include hydrochloric acid, sulfuric acid and phosphoric acids and examples of organic acids include, as those of low molecular weights, carboxylic acids, for example, aliphatic carboxylic acids, such as acetic and butyric acids, aromatic carboxylic acids, such as benzoic and phthalic acids, and oxy acids, such as tartaric and malic acids and, as those of high molecular weights, homopolymers and copolymers of polymerizable organic carboxylic acid monomers, polymerizable phenol monomers, polymerizable organic sulfonic acid monomers and polymerizable organic phosphoric acid monomers, for example, polyacrylic acid, polymethacrylic acid, polymaleic acid, polyitaconic acid, polyvinylphenol, polystyrenesulfonic acid, polyethylenesulfonic acid, poly-α-methylstyrenesulfonic acid, polyvinylphosphoric acid and polyphosphate esters.

The term “marine organism” in the present invention may be any plants or animals inhabiting in the ocean, examples including marine plants such as kelps and marine animals such as barnacles, hydroides elegans and mussels.

A process for producing a hydrogel according to the present invention will now be described. The hydrogel according to the present invention can be produced by any well-known methods. Processes for producing a hydrogel from monomers include polymerizations, such as photopolymerization, radiation polymerization, heat polymerization and catalytic polymerization with a system in which a crosslinking agent coexists. Processes for producing a hydrogel from polymers include radiation crosslinking, chemical crosslinking and physical crosslinking. In order to produce a hydrogel in which a network polymer comprising the hydrogel has acidic groups, monomers and polymers containing acidic groups will be used as raw materials. In order to produce a hydrogel in which an acidic substance exists in the gaps of a network macromolecule comprising the hydrogel, an acidic substance may physically be impregnated after producing the hydrogel or an acidic substance which does not contribute to the formation of the network macromolecule by crosslinking may be mixed into a hydrogel raw material containing monomers or polymers before producing the hydrogel.

The anti-adhesion agent for marine organisms according to the present invention can be used for the bottom of a ship, inlet channels for seawater, underwater structures such as drainage channels and tetrapods, as well as cultivation fishery and marine installations such as culture nets, buoys and stationary nets. Methods of applying the anti-adhesion agent for marine organisms include attachment of the hydrogel according to the present invention to the object by means of adhesives and direct formation of the hydrogel according to the present invention on the object.

### EXAMPLES

The present invention will now be described in detail with reference to the examples.

Preparation of Hydrogels

As synthetic macromolecular gels, anionic (negatively charged) gels—a strong electrolyte, specifically, PAMPS (poly-2-acrylamido-2-methylpropanesulfonic acid) gel and weak electrolytes, specifically, PAA (polyacrylic acid) and PMAA (polymethacrylic acid) gels were prepared.

These synthetic macromolecular gels were produced by radically polymerizing the monomers at a concentration of 1 mol/L at 60° C. for 24 hours, using methylene-bis acrylamide (MBAA) as a crosslinking agent and 0.001 mol/L of potassium persulfate (K₂S₂O₈) as an initiator. The degrees of swelling of the gels were adjusted by altering the concentration of the crosslinking agent. Each gel was produced by polymerization in a vessel made of a silicone spacer of 2 mm in thickness sandwiched by glass plates to provide a plate-like gel. After unreacted materials such as monomers were removed, the polymerized gel was equilibrated and swollen in seawater for one week and autoclaved once before use. For the PAA and PAMPS gels, in addition to the customary glass substrate (hydrophilic), a hydrophobic Teflon substrate was used for the polymerization to provide gels having graft-like slimy surfaces, because seaweeds in general are covered with viscous mucopolysaccharides on the surfaces, which may play an important role in protecting themselves from other sessile organisms. The concentrations of the crosslinking agent and initiator, the degrees of swelling of the hydrogels obtained and the proton concentrations for the Examples will be shown below.

| | monomer | crosslinking | initiator | deg. of | proton |
|---|---|---|---|---|---|
| | conc. | agent conc. | conc. | swelling | conc. |
| hydrogel | (mol/L) | (mol/L) | (mol/L) | q | (mol/L) |
| PAA | 1 | 0.002 | 0.001 | 16 | 0.87 |
| | 1 | 0.004 | 0.001 | 12 | 1.16 |
| | 1 | 0.01 | 0.001 | 5 | 2.78 |
| | 1 | 0.10 | 0.001 | 3 | 4.63 |
| PMAA | 1 | 0.004 | 0.001 | 10 | 1.16 |
| PAMPS | 1 | 0.01 | 0.001 | 16 | 0.30 |
| | 1 | 0.02 | 0.001 | 14 | 0.35 |
| | 1 | 0.04 | 0.001 | 12 | 0.40 |
| | 1 | 0.08 | 0.001 | 8 | 0.60 |

Preparation of Marine Organisms

For the Examples, a kelp was selected as a marine organism and, specifically, laminaria angusta which had been harvested during October through January at Chiyaratsunai beach, Muroran-shi, Japan was used. A mature body was cut to approximately 2 cm squares and rinsed with seawater that had been autoclaved. After briefly draining, the specimens were placed in a petri dish and kept in a cool and dark place for approximately 12 hours. Thereafter, when cold seawater was poured under a fluorescent lamp, zoospores outgrew from the specimens. After a sufficient amount of zoospores was obtained, the specimens were transferred to a beaker and nutrient seawater was added before counting the cells with a hemacytometer (approximately 150,000 cells/ml).

Experimentation

Each gel obtained above was cut to a size appropriate for a petri dish used for culture, and placed in the petri dish before adding 2 ml of the zoospores obtained above for each portion of seawater. The zoospores moved freely in the seawater and then settled to the bottom in approximately two hours. The culture was carried out in a constant temperature chamber at 15° C., using a white fluorescent lamp at a ratio of 14 hours of light to 10 hours of darkness to artificially provide a day/night condition in order to carry out the culture in a condition as natural as possible. Then the germination of zoospores and the growth of filaments were observed with an optical microscope and recorded with photographs, exchanging the nutrient seawater each time a recording was made.

Results

Anionic (Negatively Charged) Strong Electrolyte Gel (PAMPS Gel)

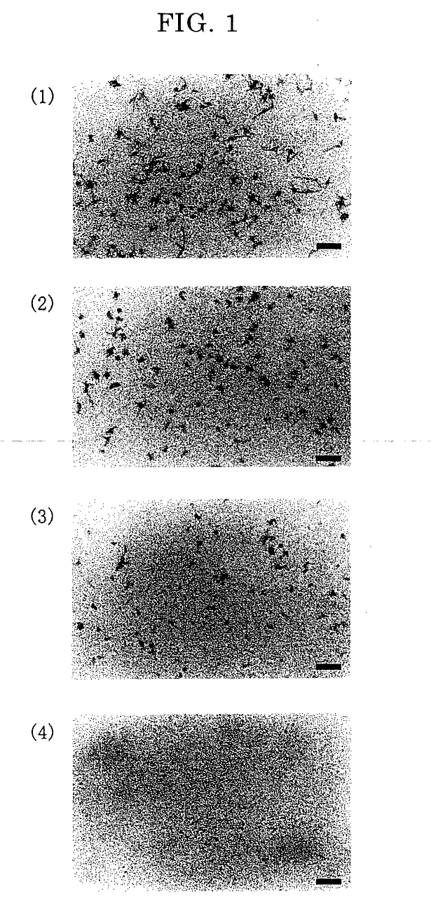

Using a PAMPS gel having strong electrolyte sulfonic groups at side chains in which the degree of swelling may greatly be altered utilizing charge repulsion by alteration of the degree of polymerization, changes of the germination rate in relation to the degree of swelling were observed. As shown in FIG. 1, (1) to (4), at 16 times the degree of swelling, the germination rate was found approximately 30%; however, the germination rate gradually decreased in accordance with the decrease of the degree of swelling and eventually decreased to 0% at 8 times the degree of swelling. It has therefore been made clear that the surface charge of a gel or the network density is closely related to the inhibition of germination.

Anionic (Negatively Charged) Weak Electrolyte Gel (PAA Gel)

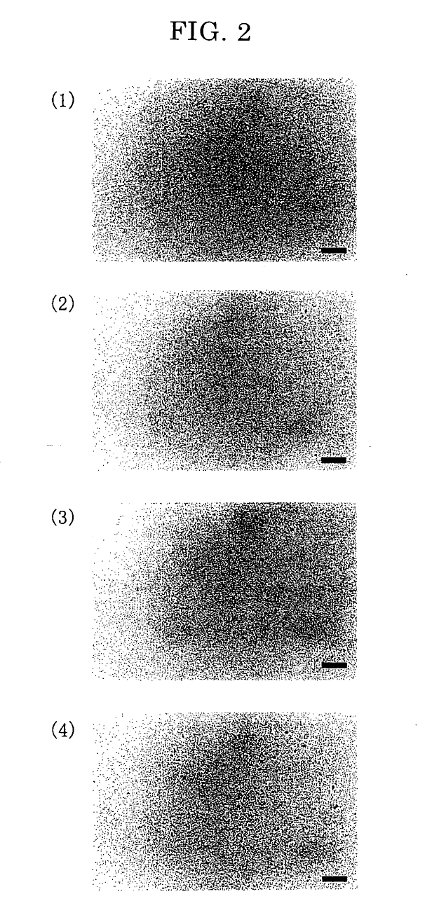

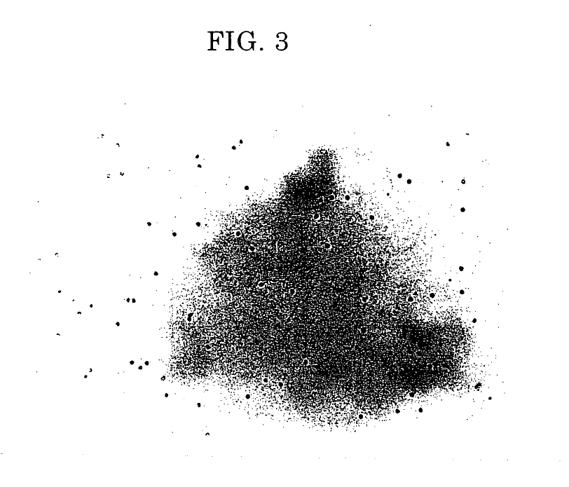

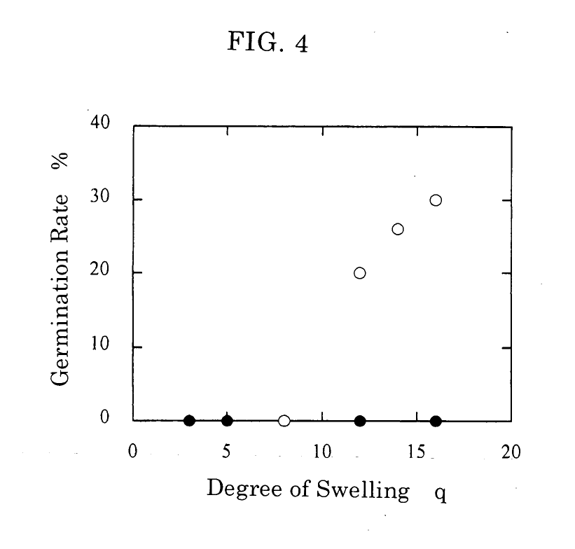

Using a PAA (polyacrylic acid) gel having weak electrolyte carboxylic groups at side chains, the dependence of the germination rate on the degree of swelling was studied. It was found that, as shown in FIG. 2, (1) to (4), at variable degrees of swelling in the range of 3 to 16, the germination rate was 0%, showing an even more effective inhibition of germination than with the PAMPS gel. This result was also observed with the PMAA (polymethacrylic acid) gel having carboxylic groups similarly to the PAA gel (refer to FIG. 3). The relationship between the germination rate of the zoospores on the PAMPS and PAA gels and the degree of swelling is shown in FIG. 4.

Zoospore Viability Study With respect to the fact that the zoospores were unable to germinate on the PAA and PMAA gels, a viability test was carried out using a dyestuff in order to investigate whether carboxylic acid was merely effective in inhibiting germination or had the effect of directly zapping the cells.

The dyestuff to be used for viability determination (Neutral Red) is known to dye the cytoplasm of a living cell but not that of a dead one. Two pairs of PAA gels on which zoospores were seeded were prepared and kept in a culture chamber for approximately 6 hours (waited until they settled to the bottom) and then one of each pair was exterminated artificially by heat treatment (70° C., 5 min.). Subsequently, 0.25 ml of 0.01% solution of Neutral Red in seawater was added dropwise to each sample and, after 5 minutes, rinsed with filtrated seawater several times before observing the dyeing by means of an optical microscope.

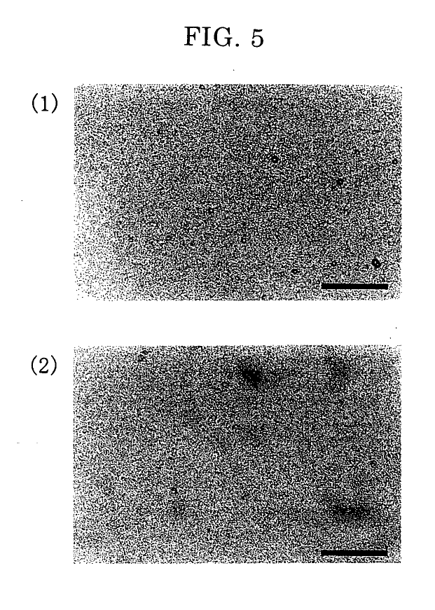

FIG. 5 shows the status of the zoospores on the PAA gels. Neither (1) the heat-treated zoospores nor (2) the untreated zoospores were found dyed and it was confirmed that all the zoospores had died in 6 hours after being seeded on the PAA gels. The dead zoospores were found adhered to the gels and it was made clear that the zoospores had recognized the PAA gels as their substrates and secreted adhesive proteins but subsequently died.

### EFFECT OF THE INVENTION

According to the present invention, adhesion of sessile marine organisms may be controlled by materials which are least harmful to the natural world and, therefore, ocean contamination by the use of highly toxic chemicals as well as damages to the ecosystem including fish and shellfish may be inhibited. In addition, when such gels are spread, coated or applied to the bottom or side of a ship, frictional resistance may effectively be reduced for a further benefit (refer to Japanese Patent Application No. 2001-13517). Also not using any consumable chemicals can expectedly reduce the financial burdens for the fishery business employees.

1. An anti-adhesion agent for marine organisms which comprises a hydrogel.

2. The anti-adhesion agent according to claim 1, wherein the hydrogel has a proton concentration of 10⁻⁴ mol/L to 5 mol/L.

3. The anti-adhesion agent according to claim 2, wherein the proton is derived from an acidic group of a network macromolecule comprising the hydrogel and/or from an acidic substance existing in the gaps in the network macromolecule.

4. The anti-adhesion agent according to claim 3, wherein the acidic group is selected from the group consisting of a carboxyl group, a hydroxyl group attached to an electron-withdrawing aromatic ring, a sulfonic group and a phosphate group.

5. The anti-adhesion agent according to claim 4, wherein the hydrogel is selected from the group consisting of homopolymerized or copolymerized macromolecular gels, chemically or physically containing in their networks, units of poly-2-acrylamido-2-methylpropanesulphonic acid (PAMPS), polyvinylphenol, polymaleic acid, polyacrylic acid (PAA) or polymethacrylic acid (PMAA).

6. The anti-adhesion agent according to any one of claims 2 to 5, wherein the acidic substance is an organic or inorganic acid.
